(19) United States
(12) Patent Application Publication
(10) Pub. No.: US 2006/0280760 A1
(43) Pub. Date: Dec. 14, 2006

(54) Assortment of personal care products having synergistic identifiers communicating products to be purchased together

(76) Inventors: Timothy Alan Scavone, Loveland, OH (US); Theresa Louise Johnson, Cincinnati, OH (US); Michael Edward Thomas, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/266,098

(22) Filed: Nov. 3, 2005

Related U.S. Application Data
(62) Division of application No. 11/150,980, filed on Jun. 13, 2005.

(51) Int. Cl. A61K 9/00; G06Q 99/00

(52) U.S. Cl. 424/400; 705/1

(57) ABSTRACT

A line of personal care products comprising a first product comprising a first identifier and a second product comprising a second identifier wherein said first identifier and said second identifier are synergistic wherein said first identifier and said second identifier together communicate that said first product and said second product should be bought and used in relation to each other.

### CROSS REFERENCE TO RELATED APPLICATION

The present application is a divisional application of co-pending U.S. application Ser. No. 11/150,980, filed on Jun. 13, 2005.

### FIELD OF THE INVENTION

The present invention is related to a line of consumer products, particularly, antiperspirants having two or more product performance characteristics comprising a sequence of designs and packaging to aid a consumer in identifying the product and/or composition so desired.

The present invention is further related to a line of personal care products comprising a first product comprising a first identifier; and a second product comprising a second identifier wherein said first identifier and said second identifier are synergistic wherein said first identifier and said second identifier together communicate that said first product and said second product should be bought and used in relation to each other.

### BACKGROUND OF THE INVENTION

Antiperspirant compositions are designed to provide effective perspiration control while also being an acceptable composition so desired by the consumer. Current merchandising displays typically shelve and arrange antiperspirant compositions according to brand. While color may play a part in the packaging, color typically only signals to the consumer a particular brand rather than a desired composition of that brand. Although there may be subtle differences in packaging amongst various product forms, consumers are typically forced to pick up each product and read the label in order to determine which particular product they wish to purchase. For example, a consumer seeking a high-efficacy invisible solid antiperspirant may reach for a product they believe is a high-efficacy invisible solid only to find that they have in their hand an original solid antiperspirant. Not only can this make for an unhappy consumer if the mistake has not been caught prior to leaving the store, but it may also be a tedious and time-consuming task. Thus, there is a need for a well-differentiated line of antiperspirant products that aids a consumer in selecting the desired level of antiperspirant efficacy.

### SUMMARY OF THE INVENTION

The present invention relates to a line of consumer products having two or more product performance characteristics, said line comprising: (a) a first product comprising at least one non-prescription strength antiperspirant and a first identifier; and (b) a second product comprising at least one prescription strength antiperspirant and a second identifier wherein said first identifier and said second identifier are different.

The present invention also relates to a line of consumer products, said line comprising: (a) a first product comprising an antiperspirant composition and a first identifier; (b) a second product comprising an antiperspirant composition and a second identifier; and (c) written and/or graphic instructions indicating that the first product should be used during a time when a person is awake and the second product should be used prior to a time when a person is asleep wherein said first identifier and said second identifier are different.

Further, the present invention relates to a method of instructing a consumer to follow an antiperspirant treatment regimen, said treatment regimen comprising applying a prescription strength, non-roll-on antiperspirant product prior to sleeping including additional written and/or graphic instructions indicating that the non-roll-on antiperspirant composition is a prescription strength formulation that should be used prior to a time when a person will be sleeping for at least about 2 hours.

Further, the present invention relates to a line of personal care products comprising a first product comprising a first identifier; and a second product comprising a second identifier wherein said first identifier and said second identifier are synergistic wherein said first identifier and said second identifier together communicate that said first product and said second product should be bought and used in relation to each other.

### DETAILED DESCRIPTION OF THE INVENTION

While the specification concludes with the claims particularly pointing and distinctly claiming the invention, it is believed that the present invention will be better understood from the following description.

As used herein, “comprising” means that other steps and/or ingredients can be added. This term encompasses the terms “consisting of” and “consisting essentially of”. The phrase “consisting essentially of” means that the invention may include additional steps and/or ingredients, but only if the additional ingredients and/or steps do not materially alter the basic and novel characteristics of the claimed invention or methods.

All percentages, parts and ratios are based upon the total weight of the topical compositions of the present invention and all measurements made are at 25° C., unless otherwise specified. All such weights as they pertain to listed ingredients are based on the active level and, therefore; do not include carriers or by-products that may be included in commercially available materials, unless otherwise specified.

The present invention involves a line of consumer products having two or more product performance characteristics. The product performance characteristic may include, but is not limited to, the product form, benefit of the product, efficacy level, fragrance level, performance level, and the like. With respect to antiperspirants, the product performance characteristic may include, but is not limited to, the product form, benefit of the product, antiperspirant efficacy, the level of odor protection, fragrance levels, hardness, and/or residue grade. The product line may comprise at least a first product and a second product wherein the two products are differentiated and identified by an “identifier” selected from the group consisting of packaging, color of packaging, shelf marking and mixtures thereof. The identifiers used to differentiate between the sequential products will also differ. Unless stated otherwise, “packaging” includes primary packaging, secondary packaging, and mixtures thereof. Additionally, the products may further include another identifier that is an indicia. As used herein, “indicia” is an identifying marking, which may include words and/or graphics describing a product in use.

The line of consumer products described herein may include a variety of consumer products including but not limited to antiperspirants, deodorants, hair care products, and personal care products including, but not limited to, body wash, body sprays, lotions, creams, and the like. The line of consumer products of the present invention may be from the same manufacturer. For example, the present invention may comprise two antiperspirant products wherein one antiperspirant product has a high antiperspirant efficacy such that it is considered to have a significantly higher level of clinically measurable wetness protection than the other antiperspirant product. The antiperspirant product having the higher level of antiperspirant efficacy may be considered “prescription strength” while the other antiperspirant composition may be considered regular or non-prescription strength. Since the line of consumer products of the present invention is not limited to only a first and second product, there may be, of course, a variety and number of products that have clinical efficacy levels in between the higher efficacy levels of antiperspirants and the lower efficacy levels of antiperspirants. As used herein, the term “prescription strength” generally refers to an antiperspirant with a significantly higher level of clinically measurable wetness protection than is available in the same product form of a non-prescription strength or regular strength formulation. The method for measuring clinical wetness protection is described in U.S. Pat. No. 6,352,688, issued to Scavone et al on Mar. 5, 2002. For example, one embodiment of the present invention may include a first non-prescription strength antiperspirant product wherein the 10-day antiperspirant efficacy is less than about 1.3 and a second prescription strength antiperspirant product that has a 10-day antiperspirant efficacy greater than about 1.3. Or, for example, one embodiment of the present invention may include a first non-prescription strength antiperspirant product wherein the 3-day antiperspirant efficacy is less than about 1.35 and a second prescription strength antiperspirant product that has a 3-day antiperspirant efficacy greater than about 1.35. Of course, one of ordinary skill in the art will also be able to determine that if the efficacy level of one product is greater than the efficacy level of another product, the product with the greater efficacy level may be considered prescription strength.

In order to aid a consumer in selecting the right level of antiperspirant protection, each product's identifier will distinguish between a higher level efficacy antiperspirant such as, prescription strength, and a lower level efficacy antiperspirant such as, regular strength (non-prescription strength), such that a consumer will be able to distinguish the products without picking the product up and reading the label. Additionally, if more than two products are used, the product identifier of the additional product or products will be distinguishable from the first, second and any other product identifier within the line. For example, the prescription strength antiperspirant may be packaged differently from the regular strength antiperspirant. Or, for example, the prescription strength antiperspirant may be packaged in a dark blue color while the regular strength antiperspirant may be packaged in a light blue color. A complimentary body spray may also be included within the line wherein the body spray may be packaged in a true blue color. Additionally, an indicia may be included on one product, some products or all products. For example, indicia may be included on the higher efficacy level antiperspirants in the form of a label that indicates that the product is of a higher efficacy level, for example “Prescription Strength”. These words may be included in one or more worldly languages and may appear on the package in one language or more than one language. Worldly languages may include, but are not limited to, English, Spanish, French, German, Chinese, Japanese, Korean, and Dutch. Or, for example, the indicia may be included on the higher efficacy level antiperspirants in the form of a label that indicates via words and/or graphics “Night” while the lower efficacy level antiperspirant may include a label that indicates via words and/or graphics “Day” to inform, encourage and/or instruct a consumer to follow a particular regimen of use.

The identifier of the line of consumer products may be synergistic such that when they are displayed or packaged in a unitary form, a consumer knows that the two products should be purchased and/or used in conjunction with each other. The synergistic identifiers together communicate to a consumer that the two or more products of the present invention should be bought and/or used in relation to each other. For example, the first identifier may be a synergistic package that physically interacts with the second identifier that may also be a synergistic package. Synergistic packaging of the present invention may physically interact by a means selected from the group consisting of interlocking, mating, co-operative assembly and connection. As used herein, “interlocking” describes a connection between two or more products wherein at least two products are locked together, for instance, a tongue and groove. As used herein, “connection” describes a connection between two or more products other than locking the at least two products together, for instance, bonding. As used herein, “mating” or “co-operative assembly” describes the interaction between two or more products wherein at least two products are in simple contact with each other such that the shape of one product conforms to the shape of the other product, for instance, hand in glove or spoon in spoon.

The synergistic identifiers may also comprise indicia wherein two or more indicia together communicate to the consumer that the two or more products should be bought and/or used in relation to each other. The indicia may be labels selected from a group of labels consisting of complimentary labels, continual labels, sequential labels, adjoining labels, and mixtures thereof. By “complimentary labels” it is meant that at least two labels are two halves that make a whole, two colors that are the same, and mixtures thereof. For example, the identifiers may be labels comprising artwork wherein the label of the first product comprises the complimentary half of the label of the second product. For example, the first product may have a label comprising a red, left half of a circle while the second product may have a label comprising a red, right half of a circle which may signal to a consumer that the two products should be bought and/or used in conjunction with each other. By “continual labels” it is meant that the combination of the first and second identifier as a whole creates a complete phrase or a complete picture. Unlike “complimentary labels”, the artwork depicted on continual labels may not be complimentary first and second halves but rather may be a first part of a picture on the first product that continues as a second part of a picture on the second product. For example, a first product may have a label comprising a picture of a lady walking a dog wherein only the lady and the upper part of a leash are seen. The second product may have a label comprising a picture of a bottom half of a leash and the dog that is being walked by the lady on the first identifier. Thus, there is a continuous picture wherein a consumer can identify the two products as those that may be bought and/or used in relation to each other. Or, for example, a first and second identifier as a whole may spell the word “Brand” wherein the first product has a label comprising the letters “Bra” and the second product has a label comprising the letters “nd” wherein a consumer can identify the products spelling a complete word as those that may be bought and/or used in relation to each other. By “sequential labels” it is meant that the products may be displayed communicating the order of use or the time of use. For example, the line of products may comprise a first product having a label comprising the word or number one, the second product having a label comprising the word or number two and a third product having a label comprising the word or number three. Or, for example, there may be a first antiperspirant product with a picture and/or at least one word indicating “Day” and a second antiperspirant product with a picture and/or at least one word indicating “Night”. By “adjoining labels”, it is meant that the first and second identifier may comprise at least one label that adheres to at least two products and indicate to a consumer that the products should be bought and/or used in relation to each other.

It is to be noted that synergistic identifiers of the present invention may not only create a system of complimentary products to be sold, bought or packaged together but it may also encourage consumers to follow a regimen of use. Additionally, the identifiers of the present invention may communicate to the consumer without the additional labor of picking up the product and reading the label.

Method of Use

The line of consumer products of the present invention may be applied topically to the underarm or other suitable area of the skin in an amount effective to reduce or inhibit perspiration wetness. Compositions of the present invention may be applied in an amount ranging from at least about 0.1 gram but no more than about 20 grams, no more than about 10 grams, or no more than about 1 gram.

The line of consumer products of the present invention may be useful as a treatment regimen, for example, treating hyperhidrosis. For example, a first antiperspirant product having a first identifier may be applied to the underarm once while awake and a second antiperspirant product having a second identifier may be applied to the underarm once while asleep. Or, for example, the regular strength antiperspirant product may be applied to the underarm once a day while awake and the prescription strength antiperspirant product may be applied to the underarm once prior to asleep. Of course, it is also suitable if the reverse method is used wherein a person applies the prescription strength while awake and the non-prescription strength prior to going to sleep. Preferably, the application while awake is considered the day, for example, after sunrise to a period prior to sundown while the sleep period is considered the night, for example, sundown to a period prior to sunrise. If this is not the case, the products should be applied according to waking and sleeping periods. Sleeping may include rest for a period of time of at least about 1 hour, at least about 2 hours, at least about 4 hours or at least about 8 hours. Additionally, written and/or graphic instructions may be included to indicate when the respective first and second products should be used.

It is also a desire of the present invention to provide a treatment regimen treating hyperhidrosis by providing a method of convincing a consumer to follow an antiperspirant treatment regimen using only the prescription strength antiperspirant prior to sleeping. Preferably, the prescription strength antiperspirant is not a roll-on; however, any other known or otherwise effective product form for providing topical application of antiperspirant or deodorant active to the desired area of the skin may be used. The prescription strength antiperspirant product of the present invention will also be non-irritating to the skin and may also be absent of aluminum chloride. If only the prescription strength non-roll antiperspirant of the present invention is used, it is desired that the product is used prior to sleeping. Sleeping may include a period of time of at least about 1 hour, at least about 2 hours, at least about 4 hours or at least about 8 hours. Additionally, written and/or graphic instructions may be included to indicate that the non-roll-on antiperspirant composition is a prescription strength formulation that should be used prior to a time where a person will be asleep.

Without being limited to the above treatment regimens, the present inventors have found that such treatment regimens may help to treat hyperhidrosis by achieving effective antiperspirant reduction or inhibition over an extended period. Efficacy levels may be evaluated according to the above disclosure. Additionally, testing procedures should be comparable to the recommended method of use. For example, if the treatment regimen instructs one to apply the product at a particular time of day, such as the evening, then the clinical methodology should also reflect this.

Product Form

The antiperspirant compositions of the present invention can be formulated as any known or otherwise effective product form for providing topical application of antiperspirant or deodorant active to the desired area of the skin. Non-limiting examples of such product forms include liquids (e.g., aerosols, pump sprays, roll-ons), solids (e.g., gel solids, invisible solids, wax solid sticks), semi-solids (e.g. creams, soft solids, lotions), and the like. For example, the antiperspirant compositions of the present invention may be semi-solids or solids.

The antiperspirant products are generally stored in and dispensed from a suitable package or applicator device. For example, the package or applicator device may be a cream dispenser with perforated application domes, etc. These packages should be sufficiently closed to prevent excessive loss of volatiles prior to application.

Method of Manufacture

The antiperspirant compositions of the present invention may be prepared by any known or otherwise effective technique, suitable for providing an anhydrous composition of the desired form and having the essential materials described herein. Many such techniques are described in the antiperspirant/deodorant formulation arts for the described product forms.

The line of consumer products of the present invention may be packaged individually in separate containers or they may be packaged together in a unitary form to be sold and bought together. Written and/or graphic instructions may be included in the separate packages or in the unitary packages to instruct a consumer when and how to use the product. Additionally, the product or products may be packaged in a secondary package wherein an outer container embodies the product disclosed therein.

### EXAMPLES

The following examples illustrate the claimed line of consumer products of the present invention having two or more product performance characteristic and differentiating identifiers. These examples, however, are not intended to be limiting thereof.

[image]

[image]

All documents cited in the Detailed Description of the Invention are, are, in relevant part, incorporated herein by reference; the citation of any document is not to be construed as an admission that it is prior art with respect to the present invention. To the extent that any meaning or definition of a term in this document conflicts with any meaning or definition of the term in a document incorporated herein by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A line of personal care products comprising:
a. a first product comprising a first identifier; and
b. a second product comprising a second identifier
wherein said first identifier and said second identifier are synergistic wherein said first identifier and said second identifier together communicate that said first product and said second product should be bought and used in relation to each other.

2. A line of personal care products of claim 1 wherein said first identifier comprises a synergistic package that physically interacts with said second identifier comprising a synergistic package.

3. A line of personal care products of claim 2 wherein the physical interaction is by a means selected from the group consisting of interlocking, mating, co-operative assembly and connection.

4. A line of personal care products of claim 1 wherein the first identifier further comprises a first indicia and the second identifier comprises a second indicia.

5. A line of personal care products of claim 4 wherein said first indicia comprises a first synergistic label and said second indicia comprises a second synergistic label.

6. A line of personal care products of claim 5 wherein said first synergistic label and said second synergistic label are selected from the group consisting of complimentary, continual, sequential, adjoining, and mixtures thereof.

7. A line of personal care products of claim 6 wherein said first synergistic label and said second synergistic label are complimentary.

8. A line of personal care products of claim 1 wherein said first identifier and said second identifier are selected from the group consisting of packaging, color of packaging, shelf markings and mixtures thereof.

9. A line of personal care products of claim 1 wherein said first identifier and said second identifier are synergistic and further communicate a regimen of use to follow.

10. A line of personal care products of claim 1 wherein the product is a hair care product.
